(19) United States
(12) Patent Application Publication
(10) Pub. No.: US 2023/0044183 A1
(43) Pub. Date: Feb. 9, 2023

(54) PURIFICATION METHOD OF PROGESTERONE

(71) Applicant: ZHEJIANG SHENZHOU PHARMACEUTICAL COMPANY LIMITED, Taizhou (CN)

(72) Inventors: Zhenping SHAO, Taizhou (CN); Rong WANG, Taizhou (CN); Bingqian WANG, Taizhou (CN); Hongfu WANG, Taizhou (CN); Chengcheng HUANG, Taizhou (CN); Lingzhi LEI, Taizhou (CN); Youfu WANG, Taizhou (CN)

(21) Appl. No.: 17/792,369

(22) PCT Filed: Feb. 25, 2021
(86) PCT No.: PCT/CN2021/077911

§ 371 Date: Jul. 12, 2022

(30) Foreign Application Priority Data
May 25, 2020 (CN) 202010451197.6

(51) Int. Cl. C07J 7/00

(52) U.S. Cl. CPC C07J 7/002

(57) ABSTRACT

The present disclosure provides a purification method of progesterone. In this method, aldehyde impurities in a progesterone crude product react with a sulfurated nucleophile to yield highly polar hydroxysulfonates and progesterone to realize solubility differentiation, and highly pure progesterone with a content higher than 99.5% and a total aldehyde impurities content lower than 0.2% is obtained through elutriation, filtration, washing a filter cake with water, and drying. The purification method provided by the present disclosure avoids the technical bottleneck of progesterone purification in the prior art, and all reagents and raw materials used during the reaction are inexpensive and easily available, with low equipment requirement and cost input; the purification process is mild and safe, conditions are easy to control, and the purification and recovery rate of the product is high; reagents used cause little environmental pollution, and particularly highly polluting chromium-containing reagents are not used.

### TECHNICAL FIELD

The present disclosure relates to the technical field of preparation process of steroids, specifically to a purification method of progesterone, and in particular to a purification method of a progesterone crude product containing aldehyde impurities.

### BACKGROUND ART

Progesterone is a natural progesterone secreted by the corpus luteum of the ovary, which has a significant morphological effect on the endometrium excited by estrogen, is necessary for pregnancy, and plays an important role in clinical medicine.

Using bisnoralcohol as a starting material, there are a plurality of methods for synthesizing progesterone.

Method A is to oxidize bisnoralcohol into bisnoraldehyde, followed by catalytic oxidation to yield progesterone. Its synthetic route is as follows:

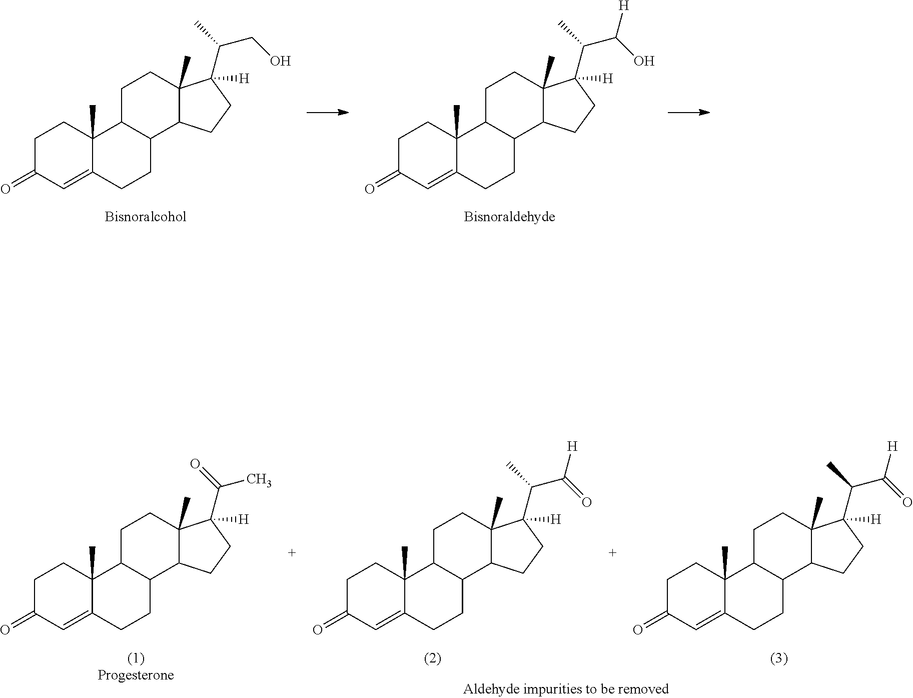 [SMILES: C[C@H](CO)[C@H]1CCC2C3CCC4=CC(=O)CC[C@]4(C)C3CC[C@@]21C ; C[C@H](CO)[C@H]1CCC2C3CCC4=CC(=O)CC[C@]4(C)C3CC[C@@]21C ; CC(=O)[C@H]1CCC2C3CCC4=CC(=O)CC[C@]4(C)C3CC[C@@]21C ; C[C@H](C=O)[C@H]1CCC2C3CCC4=CC(=O)CC[C@]4(C)C3CC[C@@]21C ; C[C@@H](C=O)[C@H]1CCC2C3CCC4=CC(=O)CC[C@]4(C)C3CC[C@@]21C]

This method is difficult to convert bisnoraldehyde completely during the catalytic oxidation. Therefore, there are a plurality of bisnoraldehyde impurity (2) and its C20 epimer impurity (3) in final product progesterone.

In Method B, bisnoralcohol is oxidized into bisnoraldehyde, the bisnoraldehyde is further reacted to produce enamine, and the enamine is catalytically oxidized to yield progesterone. Enamine intermediate in this method is also difficult to react completely, which will also be converted into bisnoraldehyde impurity (which also contains its C20 epimer). Its synthetic route is as follows:

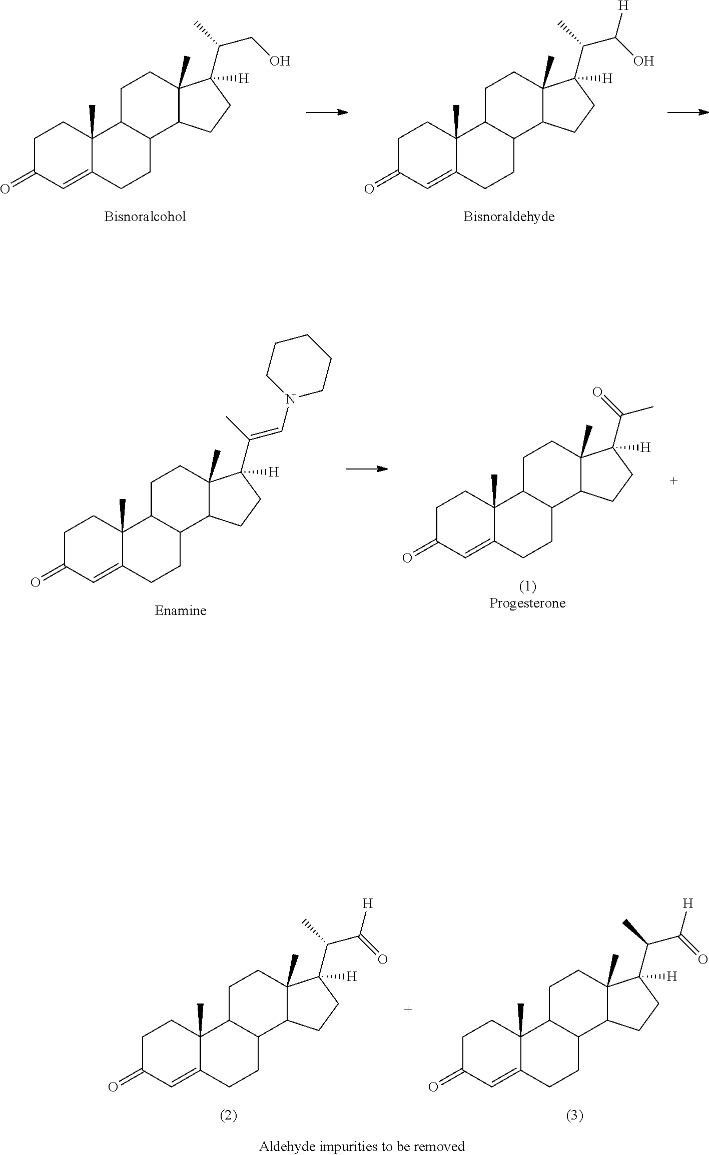 [SMILES: C[C@H](CO)[C@H]1CCC2C3CCC4=CC(=O)CC[C@]4(C)C3CC[C@@]21C ; C[C@H](CO)[C@H]1CCC2C3CCC4=CC(=O)CC[C@]4(C)C3CC[C@@]21C ; C/C(=C\N1CCCCC1)[C@H]1CCC2C3CCC4=CC(=O)CC[C@]4(C)C3CC[C@@]21C ; CC(=O)[C@H]1CCC2C3CCC4=CC(=O)CC[C@]4(C)C3CC[C@@]21C ; C[C@H](C=O)[C@H]1CCC2C3CCC4=CC(=O)CC[C@]4(C)C3CC[C@@]21C ; C[C@@H](C=O)[C@H]1CCC2C3CCC4=CC(=O)CC[C@]4(C)C3CC[C@@]21C]

Visibly, if bisnoralcohol is used as a starting material to synthesize progesterone, a plurality of bisnoraldehyde impurities will be present in the final product. Furthermore, because bisnoraldehyde impurities have physicochemical properties close to progesterone, it is difficult to or there is no way to remove them by using conventional purification methods such as recrystallization and adsorption, posing an insurmountable barrier to obtaining highly pure progesterone in industrial production. It has been tried to use a chromium-containing oxidant to oxidize bisnoraldehyde into an acid and remove aldehyde impurities by alkaline wash. However, this method is cumbersome in operation, produces a great deal of wastewater containing chromium; moreover, oxidization with oxidants may bring some new organic impurities and heavy metal impurities, which increases the difficulty of purification and is not suitable for industrial production.

As described in European Pharmacopoeia 9.0, the content of bisnoraldehydes (2) and (3) in progesterone substance on HPLC must be <0.6%, otherwise it is considered as an unqualified product. The content of bisnoraldehydes in progesterone crude product is 2 to 3%. Through general purification methods, there is no way to reduce the content of the impurity to 0.6% or below, namely, it is difficult to produce qualified progesterone substance that complies with the European Pharmacopoeia.

### SUMMARY

To solve the above problems present in the prior art, the present disclosure avoids the technical bottleneck of progesterone purification in the prior art, and provides a purification method to obtain highly pure progesterone, which is simple to operate and has a significant effect.

The objective of the present disclosure is achieved by the following technical solution:

A purification method of progesterone is disclosed, where the method has the following reaction route:

where M represents any one or more from the group consisting of H, Li, Na, and K; and

the method specifically includes the following steps:

step 1, conducting an initial reaction on a progesterone crude product containing aldehyde impurities to convert the aldehyde impurities therein into highly polar hydroxysulfonates, where the initial reaction is treated as follows:

completely dissolving the progesterone crude product containing aldehyde impurities in an organic solvent A having a volume 1 to 15 times the weight of the progesterone crude product fed, adding a sulfurated nucleophile having a volume 0.05 to 5 times the weight of the progesterone crude product fed, stirring at 20 to 60° C. to react for 0.1 to 2 h, and cooling down to −20 to 5° C. to obtain a mixture, namely an initial reaction treated product of the progesterone crude product;

alternatively,

completely dissolving the progesterone crude product in a mixed solvent of organic solvents A and B in a volume ratio of (0.5-2):1, where the organic solvent B has a volume 1 to 5 times the weight of the progesterone crude product fed; adding a sulfurated nucleophile having a volume 0.05 to 5 times the weight of the progesterone crude product fed, intensely stirring at 20 to 60° C. to react for 0.1 to 2 h, extracting a resulting reaction mixture with an organic solvent B having a volume 5 to 30 times the weight of the progesterone crude product fed, washing with water until neutral, and concentrating to obtain a mixture, namely an initial reaction treated product of the progesterone crude product;

alternatively,

adding an aqueous alkaline solution having a volume 0.1 to 5 times the weight of the progesterone crude product fed to an organic solvent A having a volume 1 to 15 times the weight of the progesterone crude product fed, passing SO₂ until pH 5 to 6, adding the progesterone crude product until completely dissolved, stirring at 20 to 60° C. to react for 0.1 to 2 h, cooling down to −20 to 5° C. to obtain a mixture, namely an initial reaction treated product of the progesterone crude product; and

step 2, adding water having a volume 0.5 to 5 times the weight of the progesterone crude product fed to the initial reaction treated product of the progesterone crude product obtained in step 1, filtering, washing a filter cake with water until neutral, and drying to obtain highly pure progesterone.

Further, the organic solvent A may be at least one selected from the group consisting of methanol, ethanol, isopropanol, n-propanol, n-butanol, tert-butanol, isobutanol, 2-butanol, tetrahydrofuran (THF), methyltetrahydrofuran, acetonitrile, N,N-dimethylformamide, and N,N-dimethyl acetamide;

further, the organic solvent B may be one selected from the group consisting of dichloromethane, chloroform, dichloroethane, toluene, and ethyl acetate;

further, the sulfurated nucleophile may be one selected from the group consisting of aqueous solutions of sodium bisulfite, potassium bisulfite, lithium bisulfite, sodium metabisulfite, potassium metabisulfite, and lithium metabisulfite, having a mass concentration of 5% to 50%;

further, the aqueous alkaline solution may be at least one selected from the group consisting of aqueous solutions of sodium hydroxide, potassium hydroxide, lithium hydroxide, sodium carbonate, potassium carbonate, lithium carbonate, sodium sulfite, potassium sulfite, and lithium sulfite, having a mass concentration of 5% to 50%.

In the method provided by the present disclosure, an aqueous alkaline solution having a volume 0.1 to 5 times the weight of the progesterone crude product fed is added to an organic solvent A having a volume 1 to 15 times the weight of the progesterone crude product fed, and SO₂ is passed until pH 5 to 6, where an objective of passing SO₂ is to react with the aqueous alkaline solution to yield the sulfurated nucleophile.

In the method provided by the present disclosure, the weight is in gram (g), and the volume is in milliliter (mL).

All raw materials related in the method provided by the present disclosure may be commercially available.

Compared with the prior art, the present disclosure has the following beneficial effects:

1) The purification method provided by the present disclosure avoids the technical bottleneck of progesterone purification in the prior art, operations are simple and convenient, with low equipment requirement and cost input;

2) in the present disclosure, purified progesterone content is higher than 99.5%, total mass yield is higher than 93%, and total aldehyde impurities content is lower than 0.2%, which meets the requirement as described in the European Pharmacopoeia 9.0, namely, the content of bisnoraldehydes (2) and (3) in progesterone substance on HPLC must be <0.6%;

3) the purification process in the present disclosure is mild and safe, conditions are easy to control, and reagents for removing aldehyde impurities are inexpensive and easily available and have very high market competitiveness in cost;

4) reagents used in the purification method provided by the present disclosure cause little environmental pollution, and particularly highly polluting chromium-containing reagents are not used, coinciding with the development trend of the green chemical industry and having excellent economic and social benefits.

### BRIEF DESCRIPTION OF THE DRAWINGS

FIG. 1 is a high performance liquid chromatogram of a progesterone crude product.

FIG. 2 is a high performance liquid chromatogram of a finished product of progesterone obtained by the purification method provided by the present disclosure.

FIG. 3 is a high performance liquid chromatogram of progesterone obtained by a common purification method.

### DETAILED DESCRIPTION OF THE EMBODIMENTS

The present disclosure will be further described below with reference to the examples, and they are not intended to limit the present disclosure.

Specific experimental procedures or conditions that are not indicated in the present disclosure shall be implemented by operations of conventional experimental procedures described in the disclosure in the art. All used reagents or equipment without specifying manufacturers may be commercially available conventional products.

### Example 1 A Purification Method of Highly Pure Progesterone

50 g of progesterone crude product (containing 2% aldehyde impurities) was fed into 50 mL of ethanol, completely dissolved, stirred with 2.5 mL of 40% aqueous sodium metabisulfite solution at 60° C. to react for 1 h, and cooled down to 5° C. to obtain an initial reaction treated product.

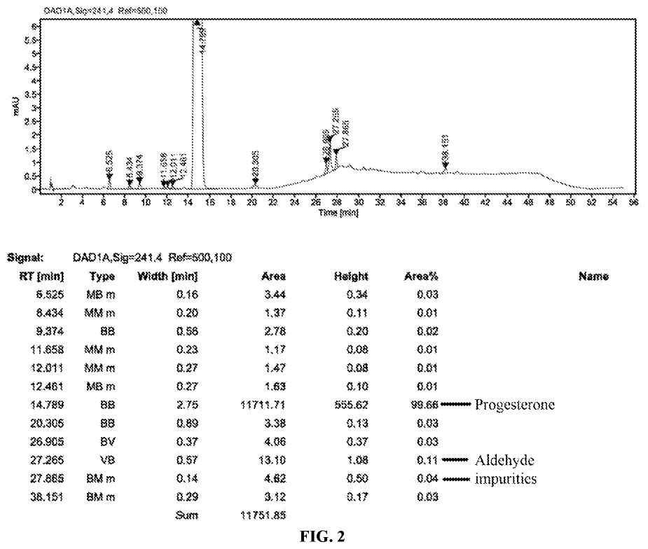

The initial reaction treated product was supplemented with 25 mL of water; after filtration, the filter cake was washed with water until neutral and dried to obtain 47.8 g of finished product of progesterone. The product yield was 95.6%, the melting point of the product was 129.0 to 130.0° C., the content on HPLC was 99.6%, and the aldehyde impurities content was <0.15%. The high performance liquid chromatogram of the finished product of progesterone is shown in FIG. 2.

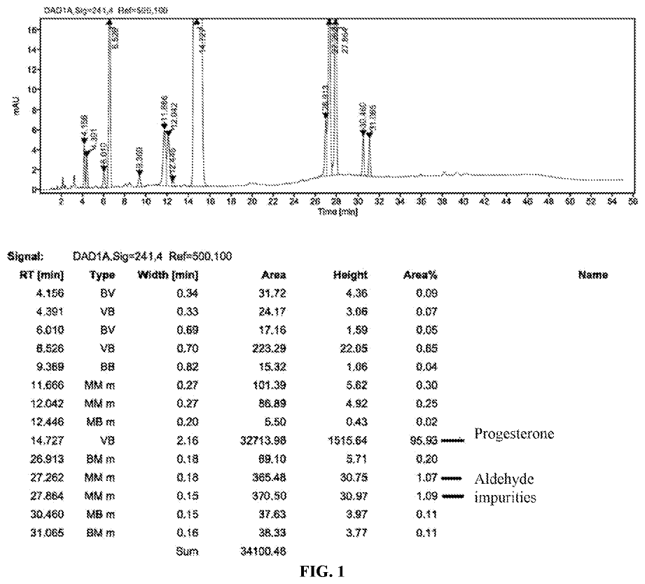

The high performance liquid chromatogram of the progesterone crude product is shown in FIG. 1.

### Example 2 A Purification Method of Highly Pure Progesterone

50 g of progesterone crude product (containing 3% aldehyde impurities) was fed into 350 mL of isopropanol, completely dissolved, stirred with 25 mL of 20% aqueous potassium bisulfite solution at 40° C. to react for 0.1 h, and cooled down to −5° C. to obtain an initial reaction treated product.

The initial reaction treated product was supplemented with 150 mL of water; after filtration, the filter cake was washed with water until neutral and dried to obtain 47.6 g of finished product of progesterone. The product yield was 95.2%, the melting point of the product was 129.1 to 130.0° C., the content on HPLC was 99.5%, and the aldehyde impurities content was <0.2%.

### Example 3 A Purification Method of Highly Pure Progesterone

50 g of progesterone crude product (containing 3% aldehyde impurities) was fed into 750 mL of tert-butanol, completely dissolved, stirred with 50 mL of 15% aqueous potassium metabisulfite solution at 20° C. to react for 2 h, and cooled down to −20° C. to obtain an initial reaction treated product.

The initial reaction treated product was supplemented with 250 mL of water; after filtration, the filter cake was washed with water until neutral and dried to obtain 47.5 g of finished product of progesterone. The product yield was 95.0%, the melting point of the product was 129.1 to 130.2° C., the content on HPLC was 99.6%, and the aldehyde impurities content was <0.15%.

### Example 4 A Purification Method of Highly Pure Progesterone

50 g of progesterone crude product (containing 3% aldehyde impurities) was fed into 100 mL of ethanol, completely dissolved, stirred with 15 mL of 35% aqueous sodium bisulfite solution at 50° C. to react for 0.5 h, and cooled down to −10° C. to obtain an initial reaction treated product.

The initial reaction treated product was supplemented with 100 mL of drinking water; after filtration, the filter cake was washed with water until neutral and dried to obtain 47.6 g of finished product of progesterone. The product yield was 95.2%, the melting point of the product was 129.0 to 130.0° C., the content on HPLC was 99.7%, and the aldehyde impurities content was <0.15%.

### Example 5 A Purification Method of Highly Pure Progesterone

50 g of progesterone crude product (containing 2% aldehyde impurities) was fed into 50 mL of dichloromethane and 25 mL of ethanol, completely dissolved, intensively stirred with 25 mL of 50% aqueous sodium bisulfite solution at 30° C. to react for 1 h, extracted with 250 mL of dichloromethane, separated, washed with water until neutral, and concentrated to obtain an initial reaction treated product.

The initial reaction treated product was supplemented with 25 mL of water; after filtration, the filter cake was washed with water until neutral and dried to obtain 47.0 g of finished product of progesterone. The product yield was 94.0%, the melting point of the product was 128.8 to 130.0° C., the content on HPLC was 99.5%, and the aldehyde impurities content was <0.2%.

### Example 6 A Purification Method of Highly Pure Progesterone

50 g of progesterone crude product (containing 3% aldehyde impurities) was fed into 250 mL of dichloroethane and 250 mL of methanol, completely dissolved, intensively stirred with 250 mL of 5% aqueous sodium metabisulfite solution at 20° C. to react for 2 h, extracted with 1,500 mL of dichloroethane, separated, washed with water until neutral, and concentrated to obtain an initial reaction treated product.

The initial reaction treated product was supplemented with 250 mL of water; after filtration, the filter cake was washed with water until neutral and dried to obtain 46.8 g of finished product of progesterone. The product yield was 93.6%, the melting point of the product was 128.9 to 130.0° C., the content on HPLC was 99.6%, and the aldehyde impurities content was <0.2%.

### Example 7 A Purification Method of Highly Pure Progesterone

50 g of progesterone crude product (containing 3% aldehyde impurities) was fed into 100 mL of toluene and 200 mL of tert-butanol, completely dissolved, intensively stirred with 100 mL of 30% aqueous potassium bisulfite solution at 60° C. to react for 0.1 h, extracted with 750 mL of toluene, separated, washed with water until neutral, and concentrated to obtain an initial reaction treated product.

The initial reaction treated product was supplemented with 100 mL of water; after filtration, the filter cake was washed with water until neutral and dried to obtain 46.5 g of finished product of progesterone. The product yield was 93.0%, the melting point of the product was 128.5 to 129.8° C., the content on HPLC was 99.5%, and the aldehyde impurities content was <0.2%.

### Example 8 A Purification Method of Highly Pure Progesterone

25 mL of 50% aqueous sodium sulfite solution was added into 50 mL of ethanol, and SO₂ was passed until pH=6; 50 g of progesterone crude product (containing 2% aldehyde impurities) was added, completely dissolved, stirred at 60° C. to react for 0.1 h, and cooled down to 5° C. to obtain an initial reaction treated product.

The initial reaction treated product was supplemented with 25 mL of water; after filtration, the filter cake was washed with water until neutral and dried to obtain 47.0 g of finished product of progesterone. The product yield was 94.0%, the melting point of the product was 128.9 to 129.8° C., the content on HPLC was 99.6%, and the aldehyde impurities content was <0.15%.

### Example 9 A Purification Method of Highly Pure Progesterone

50 mL of 10% aqueous sodium hydroxide solution was added into 300 mL of tert-butanol, and SO₂ was passed until pH=5; 50 g of progesterone crude product (containing 3% aldehyde impurities) was added, completely dissolved, stirred at 30° C. to react for 1 h, and cooled down to −5° C. to obtain an initial reaction treated product.

The initial reaction treated product was supplemented with 150 mL of water; after filtration, the filter cake was washed with water until neutral and dried to obtain 46.8 g of finished product of progesterone. The product yield was 94.0%, the melting point of the product was 128.9 to 129.2° C., the content on HPLC was 99.5%, and the aldehyde impurities content was <0.15%.

### Example 10 A Purification Method of Highly Pure Progesterone

250 mL of 5% aqueous potassium hydroxide solution was added into 750 mL of methanol, and SO₂ was passed until pH=5; 50 g of progesterone crude product (containing 2% aldehyde impurities) was added, completely dissolved, stirred at 20° C. to react for 2 h, and cooled down to −20° C. to obtain an initial reaction treated product.

The initial reaction treated product was supplemented with 250 mL of water; after filtration, the filter cake was washed with water until neutral and dried to obtain 46.9 g of finished product of progesterone. The product yield was 93.8%, the melting point of the product was 128.8 to 129.2° C., the content on HPLC was 99.5%, and the aldehyde impurities content was <0.15%.

### Comparative Example 1 A Conventional Crystallization Purification Method of Progesterone

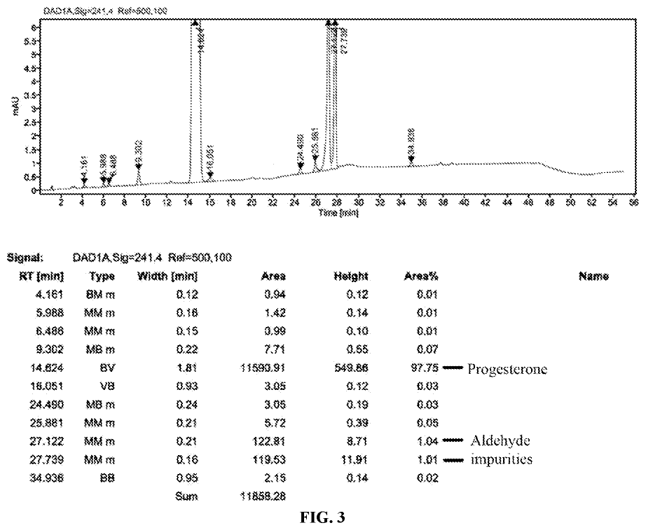

50 g of progesterone crude product (containing 2% aldehyde impurities) was added into 100 mL of ethanol, heated to 70° C. for dissolution, stirred to cool down to 0° C., filtered and dried to obtain 46 g of ethanol-crystallized substance of progesterone. The purification yield was 92.0%, the content on HPLC was 97.7%, and the aldehyde impurities content was 2%. The high performance liquid chromatogram of the resulting product progesterone is shown in FIG. 3.

Conclusion: The conventional crystallization purification method has hardly any impact on reducing the aldehyde impurities content in the progesterone crude product, and there is no way to achieve the objective of the removal of aldehyde impurities even though the method is repeated many times.

### Comparative Example 2 A Conventional Oxidation and Purification Method of Progesterone (Chromic Anhydride Method)

50 g of progesterone crude product (containing 2% aldehyde impurities) was added into 150 mL of acetone, stirred with 10 mL of 10% aqueous CrO₃ solution at 25° C. for 2 h, supplemented with 1,000 mL of water, and extracted with 200 mL of dichloromethane; the organic layer was washed once with 50 mL of 10% aqueous sodium hydroxide solution and with water until neutral, concentrated to remove dichloromethane, supplemented with 200 mL of water, cooled down to 5° C., filtered, and dried to obtain 45.0 g of progesterone. The purification yield was 90%, the content on HPLC was 95.0%, and the aldehyde impurities content was 0.8%.

Conclusion: The conventional purification method of progesterone by chromic anhydride oxidation can remove a part of aldehyde impurities from the progesterone crude product, but intensive reaction process may lead to an increase in other impurities, as well as heavy metal residues and pollution. Therefore, this method does not increase but decreases the purity of progesterone.

The above descriptions are merely the preferred specific implementations of the present disclosure, but the protection scope of the present disclosure is not limited thereto. Any modifications or substitutions easily conceived by a person skilled in the art within the technical scope disclosed in the present disclosure shall fall within the protection scope of the present disclosure.

1. A purification method of progesterone, wherein the method has the following reaction route: 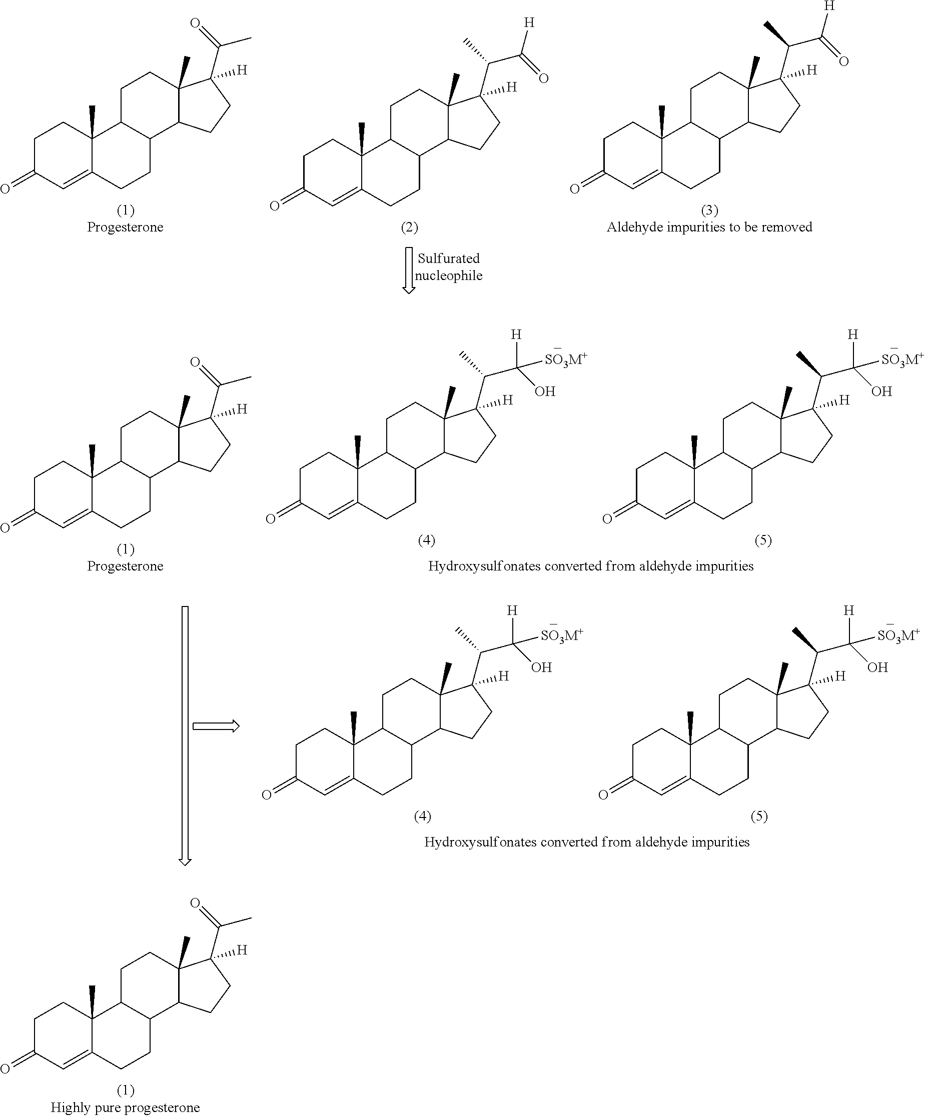
wherein M represents any one or more from the group consisting of H, Li, Na, and K; and
the method specifically comprises the following steps:
step 1, conducting an initial reaction on a progesterone crude product containing aldehyde impurities to convert the aldehyde impurities therein into highly polar hydroxysulfonates, wherein the initial reaction is treated as follows:
completely dissolving the progesterone crude product containing aldehyde impurities in an organic solvent A having a volume 1 to 15 times the weight of the progesterone crude product fed, adding a sulfurated nucleophile having a volume 0.05 to 5 times the weight of the progesterone crude product fed, stirring at 20 to 60° C. to react for 0.1 to 2 h, and cooling down to −20 to 5° C. to obtain a mixture, namely an initial reaction treated product of the progesterone crude product;
alternatively,
completely dissolving the progesterone crude product in a mixed solvent of organic solvents A and B in a volume ratio of (0.5-2):1, wherein the organic solvent B has a volume 1 to 5 times the weight of the progesterone crude product fed; adding a sulfurated nucleophile having a volume 0.05 to 5 times the weight of the progesterone crude product fed, intensely stirring at 20 to 60° C. to react for 0.1 to 2 h, extracting a resulting reaction mixture with an organic solvent B having a volume 5 to 30 times the weight of the progesterone crude product fed, washing with water until neutral, and concentrating to obtain a mixture, namely an initial reaction treated product of the progesterone crude product;
alternatively,
adding an aqueous alkaline solution having a volume 0.1 to 5 times the weight of the progesterone crude product fed to an organic solvent A having a volume 1 to 15 times the weight of the progesterone crude product fed, passing SO₂ until pH 5 to 6, adding the progesterone crude product until completely dissolved, stirring at 20 to 60° C. to react for 0.1 to 2 h, cooling down to −20 to 5° C. to obtain a mixture, namely an initial reaction treated product of the progesterone crude product; and
step 2, adding water having a volume 0.5 to 5 times the weight of the progesterone crude product fed to the initial reaction treated product of the progesterone crude product obtained in step 1, filtering, washing a filter cake with water until neutral, and drying to obtain highly pure progesterone;
wherein the organic solvent A is at least one selected from the group consisting of methanol, ethanol, isopropanol, n-propanol, n-butanol, tert-butanol, isobutanol, 2-butanol, tetrahydrofuran (THF), methyltetrahydrofuran, acetonitrile, N,N-dimethylformamide, and N,N-dimethylacetamide; the organic solvent B is one selected from the group consisting of dichloromethane, chloroform, dichloroethane, toluene, and ethyl acetate; the sulfurated nucleophile is one selected from the group consisting of aqueous solutions of sodium bisulfite, potassium bisulfite, lithium bisulfite, sodium metabisulfite, potassium metabisulfite, and lithium metabisulfite; the aqueous alkaline solution is at least one selected from the group consisting of aqueous solutions of sodium hydroxide, potassium hydroxide, lithium hydroxide, sodium carbonate, potassium carbonate, lithium carbonate, sodium sulfite, potassium sulfite, and lithium sulfite.

2. The purification method of progesterone according to claim 1, wherein the sulfurated nucleophile has a mass concentration of 5% to 50%; the aqueous alkaline solution has a mass concentration of 5% to 50%.
